SEQUENCE LISTING (10 sequences)

SEQ ID NO: 1 - DNA, 19 bp, homo sapiens
gaaggtgaaggtcggagtc

SEQ ID NO: 2 - DNA, 20 bp, homo sapiens
gaagatggtgatgggatttc

SEQ ID NO: 3 - DNA, 20 bp, homo sapiens
ccccacgacaaccgcaccat

SEQ ID NO: 4 - DNA, 20 bp, homo sapiens
cactccggcccacaaatctc

SEQ ID NO: 5 - DNA, 18 bp, homo sapiens
gtgcagagtccagcaaag

SEQ ID NO: 6 - DNA, 18 bp, homo sapiens
gataggcctcctgaaagc

SEQ ID NO: 7 - DNA, 21 bp, homo sapiens
gagacaaggacagcgaggaca

SEQ ID NO: 8 - DNA, 21 bp, homo sapiens
gtacatcaaggcacgcatcga

SEQ ID NO: 9 - DNA, 19 bp, homo sapiens
gccacaagttcagcgtgtc

SEQ ID NO: 10 - DNA, 9 bp, homo sapiens
ttcaagaga

(19) United States
(12) Patent Application Publication
(10) Pub. No.: US 2009/0214495 A1
(43) Pub. Date: Aug. 27, 2009

(54) VASCULAR ENDOTHELIAL GROWTH FACTOR-D (VEGF)-D AND FUNCTIONALLY FRAGMENTS THEREOF FOR BONE REPAIRING

(76) Inventors: Maurizio Orlandini, Siena (IT); Adriano Spreafico, Siena (IT); Salvatore Oliviero, Siena (IT)

Correspondence Address: Steinfl & Bruno, 301 N Lake Ave Ste 810, Pasadena, CA, 91101, US

(21) Appl. No.: 11/993,825

(22) PCT Filed: Jun. 9, 2006
(86) PCT No.: PCT/IT2006/000433

§ 371 Date: Feb. 12, 2008

(30) Foreign Application Priority Data
Jul. 8, 2005 (IT) RM2005A0000367

(51) Int. Cl. A61K 35/12; A61K 38/18; C12N 15/63; C12N 5/10; A61K 31/7105

(52) U.S. Cl. 424/93.21; 514/12; 435/320.1; 435/325; 514/44 R

(57) ABSTRACT

The invention refers to use of VEGF-D or fragments thereof for the preparation of a medicament for therapy and/or prevention of bone diseases, to compositions comprising the same, vectors for gene therapy, to cells expressing VEGF-D.

The invention refers to Vascular endothelial growth factor-D (VEGF-D) and functionally active fragments thereof for bone formation/reconstruction and/or bone repair. The invention refers to VEGF-D activity on osteoblasts expressing its cognate receptor VEGFR-3. The invention refers to pharmaceutical compositions for inducing or stimulating bone repair in vitro and in vivo and to methods for treatment of bone repair and/or/reconstruction in a variety of bone defects including, but not limited to, fracture, osteoporosis, vertebral or disk injury, and other bone disorders. The pharmaceutical composition may include the use of slow releasing substances, viral and non-viral delivery vehicles for the delivery of VEGF-D to target cells, which enable the cells to produce biologically active proteins that are useful for generating or healing bone.

### STATE OF THE ART

VEGF-D is a member of a family of molecules structurally related belonging to the VEGF family of angiogenic growth factors. These include VEGF (or VEGF-A), VEGF-B, VEGF-C, P1GF, and VEGF-E. These growth factors are coded by independent genes and show a limited degree of amino acid identities. Specifically, VEGF-D (GenBank® NIH NCBI accession number NM_{—}004469) shows 35% amino acid identities with VEGF (known also as VEGF-A) (GenBank® NIH NCBI accession numbers M27281; M32977), 32% amino acid identities with VEGF-B (GenBank® NIH NCBI accession numbers U43368; U43369) and 38% amino acid identities with VEGF-C (GenBank® NIH NCBI accession number X94216) 34% amino acid identities with P1GF (GenBank® NIH NCBI accession numbers X54936; S72960 ), and 33% amino acid identities with VEGF-E (GenBank® NIH NCBI accession number 84476).

VEGF-D recognizes and activates the vascular endothelial growth factor receptor (VEGFR)-2 and VEGFR-3 on blood and/or lymphatic vessels. VEGF activates VEGFR-1 and VEGFR-2, VEGF-B and P1GF activate VEGFR-1, VEGF-C activates VEGFR-2 and VEGFR-3, VEGF-E activates VEGFR-2.

VEGF-D was disclosed in the PCT publication WO97/012972, no bone and/or cartilage related activity is therein disclosed, nor mentioned.

WO2003/094617 discloses the use of VEGF (namely VEGF-A) for treating bone defeats. Though the denomination similarity, sequence data and receptor activation (as above mentioned) reveal no clear structural and functional correlation among VEGFs. Therefore it is unpredictable other functional activities of VEGF-D.

During endochondral bone formation, mesenchymal cells differentiate into chondrocytes, which secrete a cartilage template. Chondrocytes in the centers of the cartilage templates become hypertrophic and produce vascular endothelial growth factor (VEGF) that stimulates vascular invasion of the cartilage template. Upon this process, the hypertrophic chondrocytes die through apoptosis and are replaced by osteoblasts brought in from the bone collar [1,2]. The interplay between chondrocytes and osteoblasts at the growth plate determines the longitudinal growth of long bones. Osteoblasts are responsible for matrix deposition and bone mineralization. Early during their differentiation, osteoblasts express RUNX2 (also known as CBFA1), which is held inactive by Twist proteins [3]. Later, osteoblasts express the specific marker osteocalcin, which is required for bone mineralization [4].

Several growth factors expressed in the growth plate including Epidermal growth factor, members of Fibroblast growth factor family, Insulin growth factor-1, Platelet growth factor, members of Transforming growth factor-beta family, Indian hedgehog, and VEGF are involved in cell proliferation, and differentiation during bone formation [1,2,5]. Among these VEGF has been shown to play a critical role. VEGF was first identified as an angiogenic factor that is expressed in different splicing forms. It binds and activates VEGFR-1 (also known as Flt-1) and VEGFR-2 (also known as KDR or Flk1) on endothelial cells and their expression is required for vascular development and adult angiogenesis [6,7]. During skeletal development VEGF has been shown to play a role in blood vessel invasion that is essential for coupling resorption of cartilage with mineralization of the extracellular matrix and bone formation [8-10] and synergizes with BMP2 to promote bone formation and bone healing via modulation of angiogenesis [11]. VEGF is essential not only for normal angiogenesis, but also to allow normal differentiation of hypertrophic chondrocytes, osteoblasts, and osteoclasts [5, 12-18]. VEGF plays a role in bone repair. In mouse femur fractures treatment with exogenous VEGF enhanced not only blood vessel formation, but also ossification and new bone maturation, while VEGF inhibition decreased bone formation and callus mineralization [19]. Although experiments in osteoblasts demonstrated an increase of cell migration in response to VEGF [14], no functional data of the activity of VEGFRs in osteoblasts has been reported.

During mouse development, the expression of VEGF-D was detected in the periosteum/osteoblast layer of the developing vertebral column, the limb buds, and the dental mesenchyme close to the enamel epithelium [20]. As in mouse VEGF-D only recognizes murine VEGFR-3 [21] its pattern of expression suggested that VEGF-D/VEGFR-3 signaling plays a role in bone development.

VEGFR-3 has been previously shown to be involved in vascular development, lymphatic maintenance and tumor angiogenesis [22-28]. VEGF-D induces angiogenesis, lymphangiogenesis as well as metastatic spread of tumors via lymphatic vessels activating VEGFR-3 on vascular and lymphatic endothelial cells [30,36,37]. Besides playing a role in lymphatic vessels homeostasis, VEGFR-3 is implicated in the remodeling of the primary vascular network, and in reorganizing the integrity of endothelial vessels during angiogenesis [22,25-27]. On endothelial cells, VEGFR-3 signaling activates proliferation, migration, and survival [38]. The data presented demonstrate that, in addition to play a biological function in endothelial cells, VEGF-D/VEGFR-3 signaling is also implicated in osteoblast differentiation.

### DESCRIPTION OF THE INVENTION

The invention refers to the use of VEGF-D and functionally active fragments thereof for of bone development/bone formation/bone reconstructing and/or repairing. The methods described herein can be used to medical treat bone and/or cartilage defects via the systematic or local delivery of VEGF-D or fragments thereof.

One aspect of the invention is the delivery of VEGF-D. VEGF-D can be administered either by slow release preparations (biopolymer matrix, solvents), via gene delivery (DNA preparation, viral vectors, non-viral vectors), cell delivery (autologous cell implantation, heterologous (allogeneic) implantation). Furthermore, this invention is meant to include the localized expression of the therapeutic genes of interest, such as through means known to the industry, such as using tissue specific promoters, or synthetic biodegradable polymers that will localize to a specific area in the body. Additionally, the invention discloses methods to regulate the expression of the transgene through exogenously delivered molecules which will regulate the level of transgene expression. The invention also discloses methods to inhibit the expression of the transgene in one or more specific tissue types.

Also provided by the present invention is the promotion of bone formation/repair by the combination of molecules together with VEGF-D including but not limited to growth factors/active peptides/small molecules.

Also provided by the present invention is the regulation of VEGF-D for bone formation/reconstitution and/or bone repair using VEGF-D inducers, or inhibitors including antibodies, or viral vectors to express small interfering RNA.

Also provided by the present invention is the use of VEGF-D in spinal fusion surgery to promote the growth of bone to fuse the spine.

VEGF-D acts directly on osteoblasts by activating VEGFR-3 expressed in these cells. The authors investigated the involvement of the angiogenic growth factor VEGF-D and its receptor VEGFR-3 in osteoblasts. The authors showed that primary human osteoblasts express and osteoblasts of the long bones of newborn mice express VEGFR-3. Osteoblasts treated with recombinant VEGF-D respond with VEGFR-3 auto-phopshorylation, osteocalcin expression and nodule formation. The inactivation of VEGF-D activity by neutralizing antibodies or VEGFR-3 silencing inhibited VEGF-D-dependent mineralization nodule formation in osteoblasts. Data demonstrate the involvement of VEGF-D in maturation and regulation of osteoblastic activity via VEGFR-3.

### DETAILED DESCRIPTION OF THE INVENTION

The invention concerns the local or systemic delivery of synthetic human VEGF-D, or fragments essentially belonging to the central part of human VEGF-D, most in particular fragments having the amino acid sequence comprise between aa. 93-203 as deduced by the nucleotide sequence present in (GenBank® NIH NCBI accession number NM_{—}004469, by slow and/or sustained release preparations, to participate to bone formation/bone reconstructing and/or repairing.

It is a further object of the invention the use of a VEGF-D inducer for the preparation of a medicament for therapy and/or prevention of bone diseases or defects.

As used herein the term VEGF-D refers to a polypeptide having an amino acid sequence of at least 80%, preferably 85% identity to the one deduced by coding nucleotide sequence (GenBank® NIH NCBI accession number NM_{—}004469 or a fragment thereof having the biological activity of VEGF-D, or a polynucleotide sequence that can code for a polypeptide having an amino acid sequence identity of at least 80%, preferably 85% with VEGF-D or a fragment thereof having the biological activity of VEGF-D.

The term “bone defect” refers to any structural or functional bone abnormality including but not limited to fracture, bone damage, trauma, osteoporosis, osteoarthritis, vertebral or disk injury, inheritable or acquired bone disorders.

It is an object of the invention for the administration of VEGF-D or fragments thereof to activate VEGFR-3 receptor for the preparation of a medicament for therapy and/or prevention of bone defects.

Optionally, VEGF-D or fragments thereof might be administered together or in conjunction with one or more growth factors including but not limited to the BMP family of proteins (BMP-2, -4, -7, -9), the transforming growth factor family of proteins (TGF-beta-1, -2, -3), VEGF, VEGF-B, VEGF-C, P1GF, FGF.

Systemic or local delivery may be accomplished through the administration of VEGF-D or fragments thereof embedded in biopolymer matrix for injection, viral and non-viral methods and may involve the use of allogeneic, autologous, or xenogeneic mammalian cells. Both U.S. Pat. No. 5,763,416 of Bonadio et al (the “'416” patent) and U.S. Pat. No. 5,942,496 of Bonadio et al. (the “'496” patent) disclose methods for transferring nucleic acids encoding osteoinductive agents into bone cells in situ and for stimulating bone progenitor cells for the treatment of bone-related diseases and defects. The '416 and '496 patents are limited to a method for transferring a nucleic acid encoding an osteoinductive agent wherein the nucleic acid is part of a composition comprising a structural bone-compatible matrix. Appropriate matrices of the '416 and '496 patents are described as being able to both deliver the gene composition (nucleic acid) and also provide a surface for new bone growth, i.e., the matrix should act as an in situ scaffolding through which progenitor cells may migrate. However these patents do not mention mammalian cells being part of the composition. The present invention shall include the use of mammalian cells as a means to facilitate the long-term sustained expression of VEGF-D, and additionally as a mechanism to provide sufficient scaffold for the generation of new bone. Additionally, certain inventions discuss the use of synthetic materials (such as fibrin, gelatin, CaPO₄, skelite, CaCl₂, etc) as scaffolds, the present invention shall include the use of such materials in conjunction with VEGF-D as a means of generating, regenerating or healing bone.

It is another object of the invention a vector for gene therapy for the treatment and/or prevention of bone diseases comprising a sequence encoding VEGF-D or functionally active fragments thereof as above described, under the control of appropriate promoter sequences. The present invention has met the herein before described need. A method for introducing at least one gene encoding a product into at least one cell of a mammalian connective tissue for use in treating a mammalian host is provided in the present invention. This method includes employing recombinant techniques to produce a DNA vector molecule containing the gene coding for the product and introducing the DNA vector molecule containing the gene coding for the product into the connective tissue cell. The DNA vector molecule can be any DNA molecule capable of being delivered and maintained within the target cell or tissue such that the gene encoding the product of interest can be transiently or stably expressed. The DNA vector molecule preferably utilized in the present invention is either a viral or plasmid DNA vector molecule. This method preferably includes introducing the gene encoding the product into the cell of the mammalian connective tissue for therapeutic use.

The present invention is directed to a method for generating bone and/or cartilage at the site of a bone defect comprising:
a) inserting a gene encoding the VEGF-D protein or a protein sharing at least 80%, preferably 85% amino acid identity, or a functional fragment thereof having bone and/or cartilage regenerating function into a vector operatively linked to a promoter, and
b) transfecting or transducing a population of connective tissue cells in vitro with said recombinant vector, and
c) transplanting the mammalian cell into the bone defect site, and allowing the site to make the bone and/or cartilage.

In this method, the vector may be without limitation a viral vector or a plasmid vector. In addition, the connective tissue cell may be but not limited to a fibroblast, bone progenitor cell such as a stem cell, cartilage progenitor cell, or chondrocytes.

In the method above, the composition may be administered with an effective adhesive amount of bioadhesive material.

In the method above, the composition may be administered with or without an antibiotic, wherein the antibiotic may consist of the probiotic and/or bacteriocin family to reduce the possibility of infection.

In the method above, the composition may be administered in conjunction with a gelatin sheet.

In the method above, the composition may be administered in conjunction with an autologous whole blood clot, or fraction therein (such as a platelet rich plasma (PRP) fraction).

In the method above, the bone and/or cartilage is generated during early period or late period.

The composition of the invention may be administered either during or after a surgical procedure, locally at the site of the bone pathology or systemically, with a localized expression signal (such as a tissue specific promoter).

The composition may be administered to the subject one time, or multiple times. Bone defects are selected from the group consisting of bone mass loss, bone substance loss, bone structure disorders, non union disorders, defect fractures, pseudoarthrosis, bone defect states after operations, costochondritis (Tietze's syndrome), bone substance loss following failed sternal closure and healing, bone defects related to sternal dehiscence and/or mediastinitis, bone crushing injuries, bone growth disorders (such as related to dwarfism) and/or bone fractures.

The administration may also include an effective adhesive amount of bioadhesive material, preferably embedded with an anti-infective agent, or the addition of a blood clot or fraction therein (such as a platelet rich plasma “PRP” fraction), or the use of a gelatin or fibrin sheet, or the use of a structural scaffold including but not limited to CaCl₂, CaPo₄, and Skelite™.

These and other objects of the invention will be more fully understood from the following description of the invention, and the claims appended hereto.

It is another object of the invention a method for the generation of cells ex vivo to deliver VEGF-D or functionally active fragments thereof as above described.

The authors have shown that cultured primary human osteoblasts express VEGFR-3, the receptor for VEGF-D. In the long bones of newborn mice Vegfr-3 is expressed in the osteoblasts of the growing plate. The treatment of primary human osteoblasts with recombinant VEGF-D induces the expression of osteocalcin, a marker of osteoblast differentiation) and the formation of mineralized nodules in a dose-dependent manner. A monoclonal neutralizing antibody anti-VEGF-D or VEGFR-3 silencing, by lentiviral-mediated expression of VEGFR-3 small hairpin RNA (shRNA), affect VEGF-D-dependent osteocalcin expression and nodule formation. These experiments establish that VEGF-D/VEGFR-3 signaling plays a critical role in osteoblast maturation.

### BRIEF DESCRIPTION OF DRAWINGS

FIG. 1 shows the preparation of the non covalent dimers of VEGF-D. Analysis of purified VEGF-D fragment comprising aa 93-203 expressed in E. coli and refolded in vitro to generate a non covalent biologically active dimer. (A) SDS-PAGE analysis of VEGF-D purified in Ni2+ column. The purified VEGF-D in fraction 2 shows a MW of 14 kDa. (B) Separation of the purified VEGF-D in a Superdex 75 HR (Amersham Bioscience) column. This protein shows a peak at 1.46 ml indicating the presence of a protein with apparent MW of 25 kDa indicating that VEGF-D is in its dimeric form. The recombinant VEGF-D fragment comprise between aa 93-203 in its dimeric form has been used for osteoblast induction experiments.

FIG. 2 shows the expression of VEGFR-3 in osteoblasts and its activation in response to stimulation with recombinant VEGF-D. (A B) human osteoblasts were grown in complete medium and analyzed by immunofluorescence using anti-VEGFR-3 and anti-osteocalcin antibodies. (C) interference contrast of the images shown in A and B. Scale bar, 25 μm.

FIG. 3 shows the expression and autophosphorylation of VEGFR-3 in osteoblasts following VEGF-D treatment. A, cell extracts from serum-starved osteoblasts treated with VEGF-D and immunoprecipitated with anti-VEGFR-3 antibodies. The immunoprecipitation was analyzed by Western blotting with anti-phospho-Tyr antibodies, and with anti-VEGFR-3 antibodies to confirm equal loading. (B) cell extracts from serum-starved osteoblasts treated with VEGF-D were analyzed by Western blotting using anti-P-ERK1/2 and anti-ERK1/2 antibodies to confirm equal loading.

FIG. 4 shows that VEGF-D induces the formation of mineralized nodule in primary human osteoblasts. (A) Nodule formation in osteoblast cultures treated for 14 days with increasing amounts of recombinant VEGF-D. (B) Mineralized nodule formation in osteoblast cultures in the absence (NT) and presence of 25 ng/ml VEGF-D. The mineralized nodules were stained with Alizarin Red S after 14 days of treatment. Scale bar, 100 μm.

FIG. 5 shows that monoclonal antibodies anti VEGF-D inhibit nodule formation. (A) Specificity of the monoclonal antibody MAb 3.11A25. 293 cells were transfected either with a vector expressing VEGF-D or with a vector expressing VEGF-C. Immunostaining with MAb 3.11A25 demonstrated that this antibody specifically recognizes VEGF-D, but not its related factor VEGF-C. (B) Nodule formation in osteoblast cultures treated for 14 days with 25 ng/ml VEGF-D in the presence of different amounts of the MAb 3.11A25 able to inhibit the binding of VEGF-D to its receptors. NC, not correlated purified antibodies (40 μg/ml).

FIG. 6 shows that VEGF-D treatment stimulates osteocalcin production, a gene related to osteoblast differentiation. (A) Analysis of the osteocalcin and RUNX2 mRNA levels by quantitative real-time RT-PCR. RNA was extracted from primary human osteoblasts treated for 14 days with increasing amounts of VEGF-D. (B) Analysis of the osteocalcin transcripts by quantitative real-time RT-PCR. RNA was collected from osteoblasts treated for 14 days with 25 ng/ml VEGF-D and different amounts of a monoclonal antibody anti-VEGF-D, which inhibits the binding of VEGF-D to its receptors. NC, not correlated purified antibodies (40 g/ml).

FIG. 7 shows that VEGFR-3 silencing reduces the formation of mineralized nodules and osteocalcin expression in primary human osteoblasts. (A) Western blot analysis of cell extracts from cells infected with a lentiviral vector expressing unrelated (unr) or VEGFR-3 shRNA (clones D and F) grown for 14 days in complete medium. The membranes were probed with anti-VEGFR-3, anti-P-ERK1/2, anti-beta-tubulin or anti-ERK1/2 antibodies as indicated. (B) Nodule formation in control (unr) and VEGFR-3 silenced osteoblast cultures treated for 14 days with 25 ng/ml VEGF-D. (C) Analysis of the osteocalcin transcripts by quantitative real-time RT-PCR, using RNA collected from osteoblasts treated as in B. Data represent the ±SEM of 3 independent experiments each in triplicate.

FIG. 8 shows that VEGF-D is expressed in vivo in the growth plate of developing long bones of newborn mice, and VEGFR-3 is localized in osteocalcin-expressing tissues. (A) Section through ulna/radius stained with Alizarin Reds at P0, showing the developing growth plate (arrowhead). (B) Immunofluorescence analysis of a distal radius growth plate by using polyclonal anti-VEGF-D antibodies. Scale bar, 75 μm. (C, D) the growth plate of a distal radius was analyzed by immunofluorescence using respectively anti-PECAM1 and anti-VEGFR-3 antibodies. (E) Merge of C and D. Scale bar, 40 μm. F and G, the growth plate of a distal radius analyzed by immunofluorescence, using respectively anti-osteocalcin and anti-VEGFR-3 antibodies. H, merge of F and G. Scale bar, 30 μm.

The invention will be more fully understood by the following experiments as examples although they should not limit the scope of this invention.

### EXAMPLES

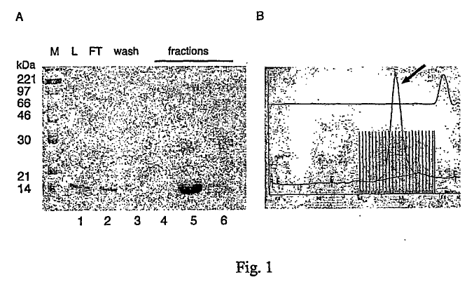

VEGF-D expressed in E. coli can be refolded in the non covalent dimer. Human VEGF-D (amino acids 90 to 203, GenBank™/EBI Data Bank accession number NM_{—}004469) were cloned by PCR from a cDNA clone containing the complete sequence of the VEGF-D gene [31] with a forward primer containing a NdeI restriction site and a reverse primer containing a SalI site. The PCR fragment was then cloned in the bacterial expression vector pET-22b (Novagen). The construct was checked by automated sequencing. VEGF-D transformed BL21-DE3 E. coli cells were grown for 3 h at 37° C. after IPTG induction, pelletted, and solubilized in 6 M guanidium chloride. VEGF-D was purified by Immobilized Metal Affinity Chromatography (IMAC) under denaturing conditions (8 M Urea) in the presence of 1 nM Tris-(2-carboxyethyl)phosphine-HCl using an AKTA purifier (Amersham Biosciences). SDS-PAGE analysis of the fractions eluted from the Ni2+ column shows a single band with molecular weight of about 14 kDa (FIG. 1A).

To obtain a VEGF-D dimer, the monomer (0,25 mg/ml) was dialyzed against: a) 6 M Urea, 0.1 M NaH₂PO₄, 10 mM Tris-HCl, 5 mM GSH, pH 8.5; b) 50 mM Tris-HCl, 5 mM NaCl, 0.5 M L-Arginine, 5 mM GSH, 1 mM GSSG, 2 M Urea, pH 8.5; c) in the same buffer as b without 2 M Urea; d) 50 mM Tris-HCl, pH8. Each dialysis was performed for 16 h. To eliminate aggregate forms, VEGF-D was loaded onto a His-TRAP affinity column in non-denaturing conditions and eluted with 250 mM imidazole. Imidazole was removed on a HiTrap desalting column and the dimer formation was checked by gel filtration using a Superdex 75 HR Column (Amersham Biosciences).

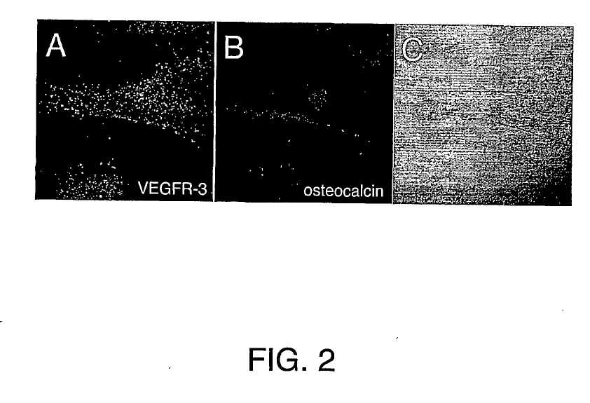

The same fraction separated in non denaturing conditions in a gel filtration column shows a elution profile of a molecule of about 28 kDa molecular weight (FIG. 2A) compatible with the formation of a non covalent dimer. This recombinant protein has been used for the biological function experiments described.

VEGF-D Activates VEGFR-3 on Primary Human Osteoblasts

Primary human osteoblasts were obtained from trabecular bone explants to analyze their expression of VEGFR-3 and response to VEGF-D. Bone samples were obtained from patients (with a mean age of 66 years) who underwent total hip replacement surgery for degenerative joint disease. After extensive washes of trabecular bone explants, small bone chips were placed in flasks with Dulbecco's modified Eagle's medium (DMEM) supplemented with 10% heat-inactivated fetal bovine serum (Life Technologies, Inc.), 2 mM L-glutamine, 50 U/ml penicillin, and 50 μg/ml streptomycin. Cultures were incubated at 37° C. in a humidified, 5% CO₂ atmosphere. For differentiation assays, osteoblasts were seeded in 6-well plates. For immunofluorescence analysis, human osteoblasts were grown on glass coverslips and, after treatment, fixed in 3% paraformaldehyde for 15 min at room temperature. For the staining of cultured human osteoblasts cells were not permeabilized. Specimens were washed twice in PBS, blocked with 1% bovine serum albumin (BSA) in PBS for 1 h at room temperature, and incubated for 1 h at 37° C. with the following primary antibodies: rat monoclonal anti-VEGFR-3 (eBioscience), goat polyclonal anti-osteocalcin (Santa Cruz Biotechnology) diluted in 1% BSA/PBS. After washing, specimens were incubated for 1 h at 37° C. with Alexa Fluor-568 or Alexa Fluor-488 secondary antibodies (Molecular Probes), and mounted in Mowiol 4-88 (Calbiochem). Fluorescent images were captured using a Leica TCS SP2 laser scanning confocal microscope.

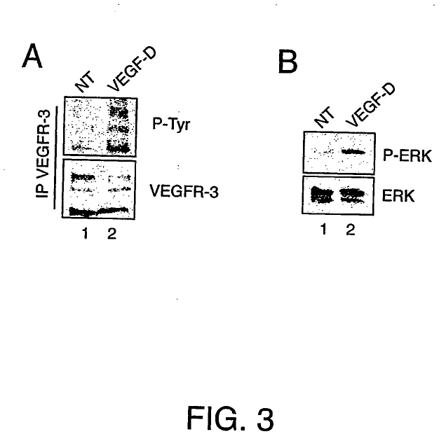

Confocal immunofluorescence analysis showed VEGFR-3 expression in primary human osteoblasts (FIG. 2). The expression of VEGFR-3 was also showed by Western blot analysis of the whole cell lysates (FIG. 3A) performing the immunoblot analysis as previously described [33].

Moreover osteoblasts were treated with VEGF-D and the whole cell lisates were immunoprecipitated with an antibody recognizing VEGFR-3 C-terminus (Santa Cruz Biotechnology) and immunoblotted with anti-phosphotyrosine antibodies (Santa Cruz Biotechnology).

Cells were stimulated for 30 min with 25 ng/ml VEGF-D at 37° C., washed with ice-cold PBS containing 0.1 mM Na₃VO₄, and lysed in 1 ml of lysis buffer (50 mM Hepes, pH 7.4, 150 mM NaCl, 1% Triton-X 100, 1.5 mM MgCl₂, 1 mM EGTA, 10 mM NaH₂PO₄, 100 mM NaF, 10 mM DTT, 1 mM Na₃VO₄, protease inhibitors, Sigma Chemical). Cells lysates were incubated on ice for 10 min, centrifuged at 10,000×g for 15 min, and the supernatants were incubated with anti-VEGFR-3 antibodies.

This analysis revealed that VEGF-D treatment induced VEGFR-3 auto-phosphorylation in osteoblasts (FIG. 3B). VEGFR-3 activation was leading to an intracellular signaling cascade as we observed that VEGF-D treatment was followed by an increase of the phosphorylation of the intracellular protein ERK1/2. These experiments demonstrate that VEGF-D recognizes and activates VEGFR-3 expressed in primary human osteoblasts.

VEGF-D Induces Osteoblasts to Form Mineralized Nodules and Induction of Osteocalcin Expression.

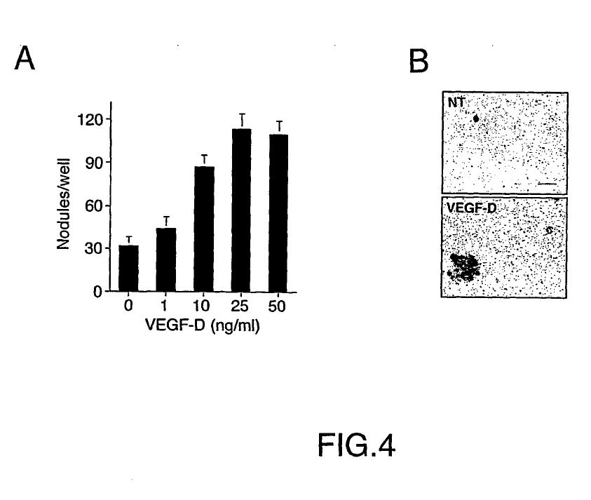

To investigate whether VEGF-D affects osteoblast differentiation, primary human osteoblasts were grown in complete medium for 14 days in the presence of different concentrations of VEGF-D. VEGF-D significantly increased the number of mineralized nodules in a dose-dependent manner (FIG. 4A). In addition, the nodules grew bigger and better mineralized than those of the control cultures that showed less and poorly mineralized nodules (FIG. 4B).

Monoclonal Antibodies Anti-VEGF-D Inhibit Nudule Formation.

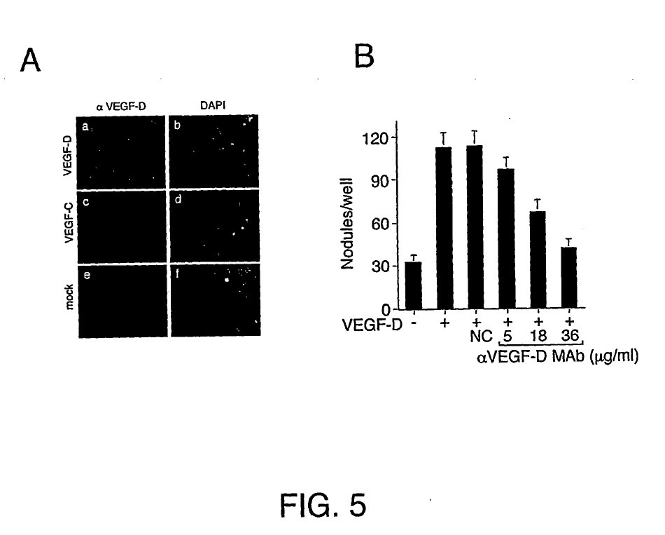

To verify whether the neutralization of VEGF-D activity affects osteoblast differentiation, we generated specific neutralizing monoclonal antibodies that recognize and immunoprecipitate VEGF-D (FIG. 5A). Monoclonal antibodies against human recombinant VEGF-D were generated using a standard fusion protocol [32]. Hybridomas were screened by enzyme-linked immuunosorbent assay. Antibody-secreting hybridomas were cloned and inoculated into pristane-primed BALB/c mice for production of ascitic fluid. The isotypes were determined using the Mouse monoclonal antibody isotyping kit (Amersham Biosciences). Antibodies were purified by affinity chromatography and characterized by immunoprecipitation and cell proliferation assays.

This antibody (MAb 3.11A25, isotype IgG2a) is able to selectively immunoprecipitate VEGF-D (FIG. 5A). The treatment of osteoblast cultures with MAb 3.11.A25 decreased nodule formation to values of untreated cells, whereas a non correlated antibody did not influence nodule formation even at high concentrations (FIG. 5B). The result demonstrates that VEGF-D activity induces osteoblasts to form mineralization nodules.

VEGF-D Induces Osteocalcin Expression in Osteoblasts.

Quantitative real-time RT-PCR analysis was performed to analyze the expression of RUNX 2, a marker of early differentiation of osteoblast and osteocalcin, a marker of late osteoblast differentiation. Total RNA was isolated from cells by the guanidinium thiocyanate method, quantified, and integrity was tested by gel electrophoresis. The gene expression analysis was performed using a LightCycler apparatus, and data were analyzed with the LightCycler software version 3.5 (Roche Applied Science). The RT-PCR reactions were set up in microcapillary tubes using the LightCycler RNA Amplification Kit SYBR Green I (Roche Applied Science) following the manufacturer's instructions. For each sample, triplicate determinations were made, and the gene expression was normalized to the amount of glyceraldehyde-3-phosphate dehydrogenase (GAPDH, Forward primer 5′-GAAGGTGAAGGTCGGAGTC-3′, SEQ ID No. 1) and Reverse primer 5′-GAAGATGGTGATGGGATTTC-3′, SEQ ID No. 2) on the same sample.

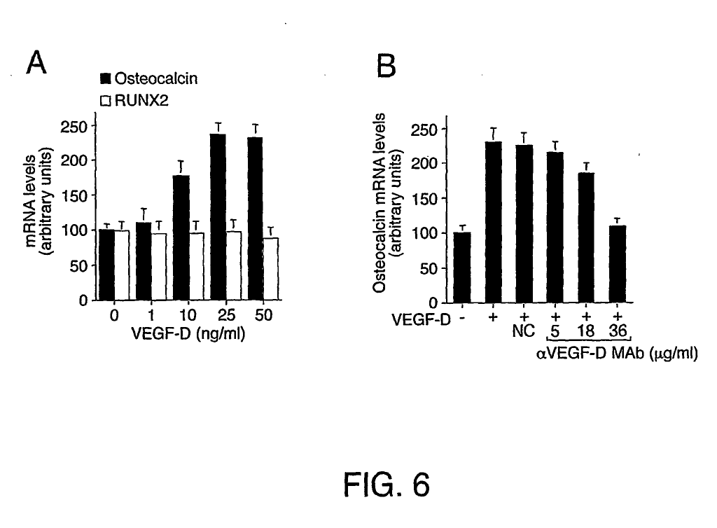

The expression of RUNX2, performed using specific primers (Forward 5′-CCCCACGACAACCGCACCAT-3′, SEQ ID No. 3 and Reverse 5′-CACTCCGGCCCACAAATCTC-3′, SEQ ID No. 4), and osteocalcin, performed using specific primers (Forward 5′-GTGCAGAGTCCAGCAAAG-3′, SEQ ID No. 5 and Reverse 5′-GATAGGCCTCCTGAAAGC-3′, SEQ ID No. 6) showed that VEGF-D treatment did not influence RUNX2 expression, while osteocalcin mRNA was significantly increased in a dose-dependent manner (FIG. 6A). Moreover, osteocalcin induction was inhibited by the anti-VEGF-D neutralizing antibody (FIG. 6B). Therefore VEGF-D induces the expression of osteocalcin, a late marker of osteoblast differentiation.

Inhibition of VEGFR-3 Expression Reduces Nodule Formation.

To investigate whether VEGF-D-dependent osteoblast differentiation acts via VEGFR-3 signaling, we generated two lentiviral vectors (clones D and F) expressing small hairpin RNA (shRNA) designed to inhibit VEGFR-3. shRNA cassette was cloned and the recombinant lentiviruses were produced as previously described [34]. Oligonucleotides coding for human VEGFR-3 and unrelated shRNA were designed to contain a sense strand 5′-GAGACAAGGACAGCGAGGACA-3′ (VEGFR-3 D clone, SEQ ID No. 7), 5′-GTACATCAAGGCACGCATCGA-3′ (VEGFR-3 F clone, SEQ ID No. 8), and 5′-GCCACAAGTTCAGCGTGTC-3′, SEQ ID No. 9 (unrelated) followed by a spacer (5′-TTCAAGAGA-3′, SEQ ID No. 10) and their reverse complementary strand followed by 5 thymidines as an RNA polymerase III transcriptional stop signal. The complementary oligonucleotides were phosphorylated, annealed, and cloned into lentiviral vector. 293 cells were transiently transfected. The lentiviruses were harvested 24 and 48 h later and filtered through 0.22-μm pore cellulose acetate filters. Recombinant lentiviruses were concentrated by ultracentrifugation for 2 hours at 50,000×g. Vector infectivity was evaluated by infecting cells with a GFP vector and titrating shRNA-expressing virus by real-time quantitative RT-PCR of a common lentiviral genome region compared with the GFP vector.

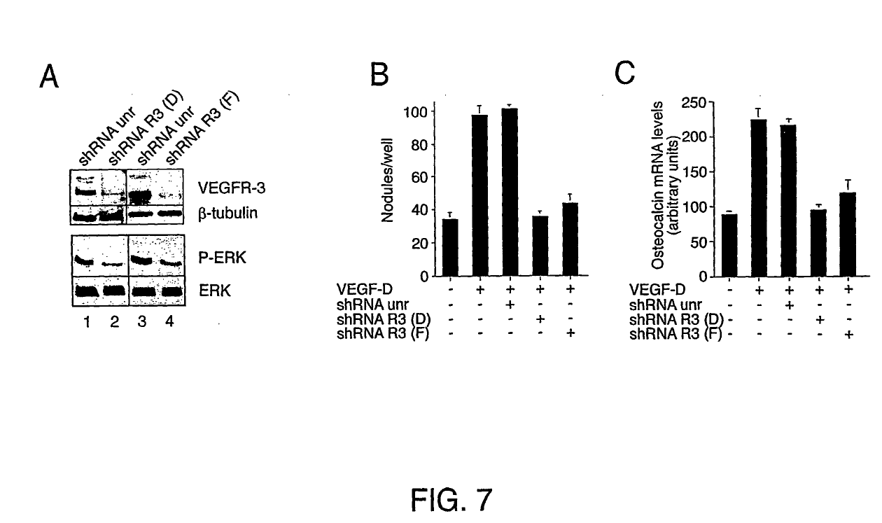

Primary human osteoblasts infected with either lentivirus expressing VEGFR-3 shRNA, but not an unrelated shRNA, showed a reduced VEGFR-3 protein expression and affected VEGF-D-dependent ERK1/2 activation (FIG. 7A). Osteoblasts knockdown for VEGFR-3, showed significant impairment of nodule formation and osteocalcin production following VEGF-D treatment (FIG. 7, BC).

Comparison of VEGF-D and VEGFR-3 Expression in the Bone of Newborn Mice.

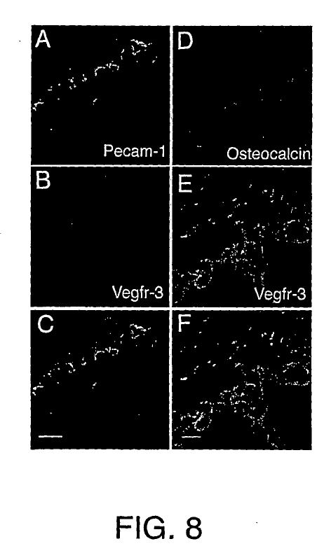

Immunohistochemical staining for murine VEGF-D performed on neonatal radius showed the expression of VEGF-D in correspondence of osteoblasts adjacent to hypertrophic chondrocytes (FIG. 8). To verify whether VEGF-D expression was compatible with a role in bone formation, we analyzed the expression of its receptor VEGFR-3, as in mouse VEGF-D only recognizes VEGFR-3 [21]. VEGFR-3 showed expression in the growth plate at the interface between the forming bone and the terminal hypertrophic chondrocytes (FIG. 8). This is a bone developing region characterized by new blood vessel formation and differentiating osteoblasts. VEGFR-3 is transiently expressed in endothelial cells during active angiogenesis [22,27,35]. Consistent with this, we observed low levels of VEGFR-3 expression in endothelial cells in the radius of new born mice, reflecting the fact that a peak of angiogenesis in the growing bone takes place before birth. Double staining between VEGFR-3 and PECAM1 (CD31), a marker of endothelial cells, revealed a partial overlapping of these signals demonstrating that VEGFR-3 is still expressed in endothelial cells at this stage (FIG. 8, C-E). A more consistent VEGFR-3 signal was observed in osteocalcin positive cells (FIG. 8, F-H). These data demonstrate that VEGFR-3 is expressed in mouse osteoblasts.

VEGFR-3 signaling plays a functional role in VEGF-D-dependent osteoblast maturation. As in mouse VEGFR-3 is expressed in osteoblasts and VEGF-D binds only this receptor [21], these results suggest that VEGF-D/VEGFR-3 signaling in bone formation is a common function between mouse and human.

The abbreviation used are: VEGF, Vascular endothelial growth factor; VEGFR, Vascular endothelial growth factor receptor; shRNA, small hairpin RNA; MAb, Monoclonal antibody; IMAC, Immobilized Metal Affinity Chromatography; DMEM, Dulbecco's modified Eagle's medium; PBS, phosphate buffered saline; BSA, bovine serum albumin; ELISA, Enzyme-Linked Immunosorbent Assay.

### REFERENCES

1. Karsenty, G. (1999) Genes Dev. 13, 3037-3051
2. Wagner, E. F., and Karsenty, G. (2001) Curr Opin Genet Dev 11, 527-532
3. Bialek, P., Kern, B., Yang, X., Schrock, M., Sosic, D., Hong, N., Wu, H., Yu, K., Ornitz, D. M., Olson, E. N., Justice, M. J., and Karsenty, G. (2004) Dev Cell 6, 423-435
4. Ducy, P., Schinke, T., Karsenty, G. (2000) Science 289, 1501-1504
5. Gerber, H. P., Vu, T. H., Ryan, A. M., Kowalski, J., Werb, Z., and Ferrara, N. (1999) Nat Med 5, 623-628
6. Ferrara, N., Gerber, H. P., and LeCouter, J. (2003) Nat Med 9, 669-676
7. Carmeliet, P. (2003) Nat Med 9, 653-660
8. Gerber, H. P., and Ferrara, N. (2000) Trends Cardiovasc Med 10, 223-228
9. Carano, R. A. D., Filvaroff, E. H. (2003) Drug Discov Today 8, 980-989
10. Aoyama, J., Tanaka, E., Miyauchi, M., Takata, T., Hanaoka, K., Hattori, Y., Sasaki, A., Watanabe, M., and Tanne, K. (2004) Histochem Cell Biol 122, 35-40
11. Peng, H., Usas, A., Olshanski, A., Ho, A. M., Gearhart, B., Cooper, G. M., and Huard, J. (2005) J Bone Miner Res 20, 2017-2027
12. Midy, V., and Plouet, J. (1994) Biochem Biophys Res Commun199, 380-386
13. Maes, C., Carmeliet, P., Moeimans, K., Stockmans, I., Smets, N., Collen, D., Bouillon, R., Carmeliet, G. (2002) Mech. Dev. 111, 61-73
14. Mayr-Wohlfart, U., Waltenberger, J., Hausser, H., Kessler, S., Gunther, K.-P., Dehio, C., Puhl, W., Brenner, R. E. (2002) Bone 30, 472-477
15. Maes, C., Stockmans, I., Moermans, K., Van Looveren, R., Smets, N., Carmeliet, P., Bouillon, R., Carmeliet, G. (2004) J Clin. Invest. 113, 188-199
16. Zelzer, E., McLean, W., Ng, Y. S., Fukai, N., Reginato, A. M., Lovejoy, S., D'Amore, P. A., and Olsen, B. R. (2002) Development 129, 1893-1904
17. Zelzer, E., Glotzer, D. J., Hartmann, C., Thomas, D., Fukai, N., Soker, S., Olsen, B. R. (2001) Mech. Dev. 106, 97-106
18. Aldridge, S. E., Lennard, T. W., Williams, J. R., and Birch, M. A. (2005) Biochem Biophys Res Commun 335, 793-798
19. Street, J., Bao, M., deGuzman, L., Bunting, S., Peale, F. V., Jr., Ferrara, N., Steinmetz, H., Hoeffel, J., Cleland, J. L., Daugherty, A., van Bruggen, N., Redmond, H. P., Carano, R. A., and Filvaroff, E. H. (2002) Proc Natl Acad Sci USA 99, 9656-9661
20. Avantaggiato, V., Acampora, D., Orlandini, M., Oliviero, S., and Simeone, A. (1998) Mech. Dev. 73, 221-224.
21. Baldwin, M. E., Catimel, B., Nice, E. C., Roufail, S., Hall, N. E., Stenvers, K. L., Karkkainen, M. J., Alitalo, K., Stacker, S. A., and Achen, M. G. (2001) J Biol Chem 276, 19166-19171
22. Dumont, D. J., Jussila, L., Taipale, J., Lymboussaki, A., Mustonen, T., Pajusola, K., Breitman, M., and Alitalo, K. (1998) Science 282, 946-949
23. Veikkola, T., Jussila, L., Makinen, T., Karpanen, T., Jeltsch, M., Petrova, T. V., Kubo, H., Thurston, G., McDonald, D. M., Achen, M. G., Stacker, S. A., and Alitalo, K. (2001) Embo J 20, 1223-1231
24. Makinen, T., Jussila, L., Veikkola, T., Karpanen, T., Kettunen, M. I., Pulkkanen, K. J., Kauppinen, R., Jackson, D. G., Kubo, H., Nishikawa, S., Yla-Herttuala, S., and Alitalo, K. (2001) Nat Med 7, 199-205
25. Partanen, T. A., Alitalo, K., and Miettinen, M. (1999) Cancer 86, 2406-2412
26. Valtola, R., Salven, P., Heikkila, P., Taipale, J., Joensuu, H., Rehn, M., Pihlajaniemi, T., Weich, H., deWaal, R., and Alitalo, K. (1999) Am J Pathol 154, 1381-1390
27. Kubo, H., Fujiwara, T., Jussila, L., Hashi, H., Ogawa, M., Shimizu, K., Awane, M., Sakai, Y., Takabayashi, A., Alitalo, K., Yamaoka, Y., and Nishikawa, S. I. (2000) Blood 96, 546-553
28. Achen, M. G., Williams, R. A., Baldwin, M. E., Lai, P., Roufail, S., Alitalo, K., and Stacker, S. A. (2002) Growth Factors 20, 99-107
29. Frediani, B., Spreafico, A., Capperucci, C., Chellini, F., Gambera, D., Ferrata, P., Baldi, F., Falsetti, P., Santucci, A., Bocchi, L., Marcolongo, R. (2004) Bone 35, 859-869
30. Marconcini, L., Marchiò, S., Morbidelli, L., Cartocci, E., Albini, A., Ziche, M., Bussolino, F., and Oliviero, S. (1999) Proc. Natl. Acad. Sci. USA 96, 9671-9676
31. Rocchigiani, M., Lestingi, M., Luddi, A., Orlandini, M., Franco, B., Rossi, E., Ballabio, A., Zuffardi, O., and Oliviero, S. (1998) Genomics 47, 207-216
32. Campbell, G. H., Miller, L. H., Hudson, D., Franco, E. L., and Andrysiak, P. M. (1984) Am J Trop Med Hyg 33, 1051-1054
33. Orlandini, M., Semboloni, S., and Oliviero, S. (2003) J Biol Chem 278, 44650-44656
34. Zippo, A., De Robertis, A., Bardelli, M., Galvagni, F., and Oliviero, S. (2004) Blood 103, 4536-4544
35. Witner, A. N., van Blijswijk, B. C., Dai, J., Hofman, P., Partanen, T. A., Vrensen, G. F., and Schlingemann, R. O. (2001) J Pathol 195, 490-497
36. Achen, M. G., Jeltsch, M., Kulck, E., Makinen, T., Vitali, A., Wilks, A. F., Alitalo, K., and Stacker, S. A. (1998) Proc. Natl. Acad. Sci. USA 95, 548-553
37. Stacker, S. A., Caesar, C., Baldwin, M. E., Thornton, G. E., Williams, R. A., Prevo, R., Jackson, D. G., Nishikawa, S., Kubo, H., and Achen, M. G. (2001) Nat Med 7, 186-191.
38. Salameh, A., Galvagni, F., Bardelli, M., Bussolino, F., and Oliviero, S. (2005) Blood 106, 3423-3431
39. LeCouter, J., Moritz, D. R., Li, B., Phillips, G. L., Liang, X. H., Gerber, H. P., Hillan, K. J., and Ferrara, N. (2003) Science 299, 890-893

1. A method for therapy and/or prevention of a bone disease or defect in a subject, the method comprising
administering to the subject an effective amount of VEGF-D protein, a protein sharing at least 80% amino acid identity with the VEGF-D protein, or a functional fragment thereof.

2. A method for therapy and/or prevention of a bone disease or defect in a subject, the method comprising
administering to the subject an effective amount of VEGF-D protein, a protein sharing at least 85% amino acid identity with the VEGF-D protein, or a functional fragment thereof.

3. A method for the therapy and/or prevention of a bone disease or defect in a subject, the method comprising
administering to the subject an effective amount of a VEGF-D inducer.

4. The method according to claim 1, wherein the VEGF-D is the human VEGF-D, as encoded by the nucleotide sequence present in GenBank NIH, N^{o} NM_{—}004469.

5. The method according to claim 1, wherein the functional fragment of VEGF-D belongs essentially to the central portion of the protein.

6. The method according to claim 5, wherein the fragment has essentially the amino acid sequence 93-203 as deduced by the nucleotide sequence present in GenBank NEH, N^{o} NM_{—}004469.

7. The method according to claim 1, wherein the amino acid sequence comprises a C-terminal fusion protein.

8. The method according to claim 7 where in the fusion protein consists of a fluorescent tag.

9. The method according to claim 7 wherein the fusion protein consists of histidine residues and is between 1 and 20 amino acids in length.

10. The method according to claim 7 wherein the fusion protein is an osteogenic or chondrogenic protein such as TGF-betal, BMP-2, -4, -7, -9, or OP-I.

11. A method for treatment and/or prevention of a bone and/or cartilage disease or defect comprising the step of administering an effective amount of VEGF-D, a protein sharing at Least 80% amino acid identity with the VEGF-D protein, a functional fragment thereof, or a VEGF-D inducer.

12. A vector for gene therapy for the treatment and/or prevention of bone diseases or defects comprising a sequence encoding VEGF-D, a protein sharing at least 80% amino acid identity with the VEGF-D protein, a functionally active fragment thereof, or a VEGF-D inducer, under the control of appropriate promoter sequences.

13. The vector according to claim 12 selected from the group consisting of adenoviral vectors, adeno-associated viral vectors, retroviraL vectors, lentivirus and herpes simplex viral vectors.

14. The vector according to claim 12 containing tissue specific promoters to restrict expression of the transgene to one or more tissue types.

15. The vector according to claim 12 wherein the vector contains a promoter or promoters in which expression can be regulated up or down.

16. The vector according to claim 12, wherein the vector is a plasmid vector.

17. A mammalian cell transformed and expressing the vector according to claim 12.

18. The mammalian cell according to claim 17 wherein said mammalian cell consists of one or more of the following: bone marrow cells, connective tissue cells, stem cells, muscle cells and/or chondrocytes or an appropriate mixture thereof.

19. The mammalian cell according to claim 18, wherein said mammalian cell is a fibroblast.

20. The mammalian cell according to claim 18, wherein said mammalian cell is an osteoblast.

21. The mammalian cell according to claim 18, wherein said connective tissue cell is a bone progenitor cell.

22. A method of cell-mediated gene therapy for the treatment and/or prevention of bone diseases or defects, the method comprising administering mammalian cells transformed with the vector according to claim 12, the method further comprising:
a. Inserting a gene encoding the VEGF-D protein or a protein sharing at least 80% amino acid identity with VEGF-D protein or functional fragments thereof having bone and/or cartilage regenerating function into a vector operatively Linked to a promoter,
b. Transfecting or transducing in vitro a mammalian cell population according to claim 17 with said recombinant vector; and
c. Transplanting the transfected mammalian cell into the bone defect site, and allowing the defect site to make the bone and/or cartilage.

23. A method for the therapy and/or prevention of a bone disease or defect in a subject, the method comprising
administering to the subject an effective amount of mRNA coding for the VEGF-D protein, a protein sharing at least 80% amino acid identity with VEGF-D protein or a functional fragment thereof having bone and/or cartilage regenerating function, the effective amount in a medicament to be directly injected to the site of bone repair.

24. The method according to claim 2, wherein the VEGF-D is the human VEGF-D, as encoded by the nucleotide sequence present in GenBank NIH, N^{o} NM_{—}004469.

25. The method according to claim 2, wherein the functional fragment of VEGF-D belongs essentially to the central portion of the protein.

26. The method according to claim 25, wherein the fragment has essentially the amino acid sequence 93-203 as deduced by the nucleotide sequence present in GenBank NEH, N^{o} NM_{—}004469.

27. The method according to claim 2, wherein the amino acid sequence comprises a C-terminal fusion protein.

28. The method according to claim 27 where in the fusion protein consists of a fluorescent tag.

29. The method according to claim 27 wherein the fusion protein consists of histidine residues and is between 1 and 20 amino acids in length.

30. The method according to claim 27 wherein the fusion protein is an osteogenic or chondrogenic protein such as TGF-betal, BMP-2, -4, -7, -9, or OP-I.
